(19) United States
(12) Patent Application Publication
(10) Pub. No.: US 2008/0318788 A1
(43) Pub. Date: Dec. 25, 2008

(54) Microcapsule

(76) Inventor: Allan Kunamoney Nadian, York (GB)

Correspondence Address: NIXON & VANDERHYE, PC, 901 NORTH GLEBE ROAD, 11TH FLOOR, ARLINGTON, VA, 22203, US

(21) Appl. No.: 11/795,238

(22) PCT Filed: Jan. 19, 2006
(86) PCT No.: PCT/GB2006/000161

§ 371 Date: Apr. 3, 2008

(30) Foreign Application Priority Data
Jan. 19, 2005 (GB) 0501060.8

(51) Int. Cl. A01N 59/16; A61K 9/50; A01P 21/00; A61K 31/235; A61K 31/695; A61K 8/11; A01N 25/28; A01N 55/10

(52) U.S. Cl. 504/187; 424/490; 424/494; 424/495; 424/492; 424/497; 514/510; 514/63; 424/401; 504/116.1; 504/193

(57) ABSTRACT

A microcapsule comprising an active component encapsulated therein, and comprising a particulate matter located in a wall thereof to render the wall permeable. Such microcapsules can be used in a variety of applications including agrochemical applications, which are also described and claimed.

The present invention relates to microcapsules, which have a permeable wall, to their uses for instance in agrochemical, cosmetic, veterinary and pharmaceutical formulations, as well as to methods for producing them.

Microcapsules have been found to be a very effective tool for aiding the delivery of active components such as chemical and biological substances to a target environment. In particular they have been found to be useful delivery vehicles for chemicals and biological substances. For instance, they can be manufactured to release their contents only under suitable conditions of pH, temperature or moisture etc.

Problems may occur however on storage or in use, due to degradation of the active component as a result of exposure to U.V. radiation. These problems occur particularly where the active component is U.V. labile, as many pharmaceutical and agrochemical substances are. In the case of agrochemicals, the problem may be aggravated by the fact that in use, the compounds may be exposed to high levels of U.V. radiation. U.V. protectants such as benzophenones: 2-hydroxy-4-n-octoxybenzophenone and 2,2′-dihyroxy-4,4′-dimethoxybenzophenone; benzotriazoles: 2-(2-hydroxy-5′-methylphenyl)-benzotriazole and 2-(3′,5′-diallyl-2′-hydroxyphenyl)benzotriazole; and free radical scavengers: bis(2,2,6,6-tetramethyl-4-piperidyl)sebecate and 8-acetyl-3-dodecyl-7,7,9,9-tetramethyl-1,3,8-triazaspiro(4.5)decane-2,5-dione are known, but may not be sufficient to provide adequate protection for the compounds under these circumstances.

Other issues may arise in relation to the detection of formulations once applied to a target. For instance, in the case of a topically applied medication, or an agrochemical applied by spraying techniques, it may be difficult to see whether adequate or complete coverage has been achieved.

The present invention provides an improved microcapsule.

According to a first aspect of the present invention there is provided a microcapsule having a permeable wall, said microcapsule comprising an active component encapsulated therein, and a particulate matter located in a wall thereof to render the wall permeable.

As used herein, the term “particulate matter” includes any small particles, for instance, microparticles such as microspheres, and nano particles (whose dimensions are less than 1 μm).

In particular, the invention relates to microcapsules which are comprised of a material which is generally impermeable under most conditions such that the active component may be contained within the microcapsule. However, the presence of particulate matter such as nano particles or microspheres located in a wall thereof, renders the wall permeable.

This permeability may be caused in various ways, for example a nano particle or microsphere may act as a wick allowing the active component to move out of the microcapsule by capillary action, or may instead or additionally allow the active component to move out of the microcapsule by some other action.

Microcapsules of this type advantageously allow for the controlled delivery of a substance encapsulated within the microsphere and are particularly useful where a slow release is required.

The particulate matter can be of any suitable material, which renders the wall permeable and is compatible with the other components, and in particular the wall of the microcapsule. The particulate matter is suitably insoluble in conditions in which the microcapsule is to be stored or used.

The particles of the particulate matter are suitably of sufficient size to ensure that when positioned in the wall of the microcapsule, the microcapsule is rendered permeable. Since the properties of the wall may vary, the size and type of the particles will need to be selected to be compatible with the type of microcapsule being used. Most suitably the particles of the particulate matter are of a size, which ensures that the particles traverse the wall of the microcapsule. Suitably the particles are less than 30 μm, preferably between 0.10 and 20 μm, more preferably between 0.10 and 10 μm and most preferably have an average diameter of 0.40 micro meters. In a particular embodiment, the particles of the particulate matter are nano particles.

Suitable particulate matter include inorganic particles such as metals, for instance titanium, iron, copper, silver, gold, lead, tin, aluminium, or insoluble salts thereof, including metal oxides. A particular example of such a particle is titanium dioxide.

Alternatively, the particles of the particulate matter may be of an insoluble polymeric material. Suitable materials include insoluble polymers such as insoluble polysaccharides, polyacrylates, polymethacrylates, polyacrylic acids, polymethacrylic acids, polyalkylenes such as polythenes, polyurethanes or polystyrenes, or copolymers of these. Particularly suitable polymers include polysaccharides such as cellulose or derivatives thereof, such as alkyl cellulose, for instance ethyl cellulose.

Suitably at least some of the particles of the particulate matter are coated with a material that further enhances permeability through the microcapsule. A particular example of such a material is silica. In particular, where the particles are inorganic particles as described above, a silica coating has been found to be particularly useful in enhancing the permeability inducing properties of the particles. This may be due to some wicking effects. Thus, in a particular embodiment, the particulate matter comprises silica coated titanium dioxide, such as the material available commercially as Ti-Pure®, and in particular Ti-Pure® R-931 (DuPont, Wilmington, Del., USA).

As the amount of particulate matter is increased the permeability of the microcapsule has been found to increase. Preferred ratios of particulate matter to microcapsule wall material are from 1:2 to 4:1. Most preferably the ratio of particulate matter to microcapsule wall material is 1:1.

The presence of particulate matter, for example of titanium dioxide, and in particular silica coated titanium dioxide particles, such as Ti-Pure® R-931 may have an additional advantage of inhibiting aggregation of the dispersed droplets during production of the microcapsules. Sometimes during the preparation of the microcapsules, especially capsules below 50 microns, the dispersed droplets are encapsulated as aggregates resulting in bigger capsules. The presence of particulate matter may inhibit this aggregation enabling discrete small microcapsules to be formed. These smaller microcapsules may be preferred as they can be easier to apply to a plant or an animal by spraying, as they do not clog up the nozzle of any spraying device.

The permeability of the microcapsule may further be enhanced by combining the particulate matter with a leachable material, which is at least partially leached out of the particulate matter, either prior to use or subsequent to incorporation into the microcapsule. This appears to enhance the permeability of the microcapsules in certain circumstances.

Particular examples of suitable leachable materials include certain polymers, for instance copolymers of methacrylates and methacrylic acid. A particular example is a copolymer of cationic dimethylaminoethylmethylmethacrylate and neutral methacrylic acid ester, for instance as available commercially as Eudragit® E100 (Degussa, Dusseldorf, Germany). Eudragit® E100 is a copolymer of cationic dimethylaminoethylmethyl methacrylate and neutral methacrylic acid ester having the following structure:—

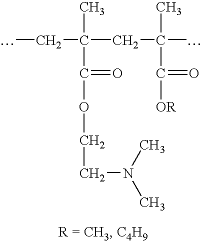

This is suitably incorporated into the wall of the impermeable microcapsules as described above. It can be leached using hydrochloric acid, in particular 1M HCl using conventional conditions. Typically the microcapsules were suspended in aqueous 1M HCl with agitation at room temperature for 18 hours to leach the Eudragit E100 from the capsule wall. The capsules were subsequently washed thoroughly and resuspended in water.

In a preferred embodiment a silica coated particle, for example, a titanium dioxide particle such as Ti-Pure®R-931 is located in a wall of the microcapsule to render said wall permeable.

In a preferred embodiment, the microsphere is combined with or further comprises a dye. The presence of the dye, in particular one that absorbs U.V. light protects the active component from degradation. Additionally or alternatively, it may also provide a means for detecting the microsphere after application. Additionally or alternatively it may reduce any phytotoxic effects of the microcapsule or the particulate matter.

The dye is preferably incorporated within or located on the surface of the microcapsule but may instead be free from the microcapsules, for example in a solution surrounding the microcapsules.

As used herein, the term “dye” refers to any material which can be detected visually, and/or which absorbs UV radiation. Suitably it is able to colour or stain material it comes into contact with.

The dye is suitably one that allows visible monitoring of the application of such microcapsules to, for example, the surface of a plant, or the skin of a human or animal. For example, applying a microcapsule as described above which contains, for example an encapsulated agrochemical and a dye would give a visual indication as to which plants have been treated and which plants have not been treated therefore ensuring that none are missed or repeated by accident.

Formulations of this type, for example, a pesticide formulation, a sun tan lotion formulation or a topical medicine formulation, when applied, would leave a mark on the skin of the animal such as human to whom it is applied, giving a visual indication of the areas of skin to which the formulation has and has not been applied.

Most preferably the dye is an environmentally acceptable dye. In general, this will mean any dye, which is permitted in food, drug, cosmetic and pesticide formulations by the relevant government bodies. Thus such dyes are either agriculturally, pharmaceutically or veternarily acceptable dyes.

Preferably the dye is Acid Orange 51, Acid Orange 63, Acid Orange 74, Bismark Brown R, Bismark Brown Y, Bromocresol Green, Chlorophenol Red, Chrysoidin, Congo Red, m-crestol Purple, Crocein Orange G, Darrow Red, Direct Black 22, Ethyl Orange, Ethyl Red, Mordant Brown 1, Mordant Brown 4, Mordant Brown 33, Mordant Brown 48 or Chocolate Brown, or combinations thereof. However, a silver stain may be employed when this is not incompatible with the end use of the formulation.

In a particular embodiment, the dye is any dye which has a U.V. absorption spectrum which is similar to that of Bismark Brown. By “similar” it is meant that the peak absorption occurs at approximately the same wavelength as the peaks of the Bismark Brown spectrum, and/or is a dye which appears in Table 1 below.

Most preferably the dye is Chocolate Brown. (Brown 3, CI-20285, E155, WS Simpson, London, UK), which has the following chemical structure:—

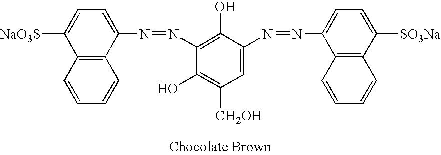

Chocolate Brown is particularly preferred for use in microcapsules comprising titanium dioxide. Titanium dioxide is at least partially phytotoxic to some plants, and therefore the use of a dye, which is capable of reducing the phytoxic effects of the titanium dioxide, is preferable.

The active component is encapsulated within the microcapsule, but may additionally be located on the surface of the microcapsule and/or be present in a solution surrounding the microcapsule.

The active component may comprise a living or non-living component. Suitable living components are bacteria, nematodes, viruses or fungi, which may or may not be inactivated or attenuated. Preferably the active component is a non-living component, such as a chemical compound, or a reagent that is derived from a living component, for example an immunogen such as a polypeptide or protein, as well as killed microorganisms such as heat or chemically killed bacteria and/or viruses

The active components are suitably agrochemical, pharmaceutical, cosmetic or veterinary reagents.

Suitable cosmetic reagents may include perfumes and other fragrances.

In a particular embodiment the active component is other than an anti-bacterial component.

Most preferably the microcapsule encapsulates an agrochemical, which herein shall be taken to include pesticides such as insecticides, acaricides, fungicides and herbicides, as well as plant growth regulators and fertilizers. Such microcapsules would be very useful in the field of agriculture and horticulture where spraying with such agrochemicals is very common.

Most preferably the agrochemical is a pesticide for example, a fungicide and especially an insecticide or acaricide. The agrochemical may be photo labile, in the sense that it is unstable or degrades over time, when exposed to U.V. light.

Suitable agrochemicals are naphthoquinone derivatives.

The term “naphthoquinone derivative” shall be taken herein to mean any agriculturally useful compound containing a naphthalene core, substituted by two oxo groups, and suitably one or more further substitutents. In particular, they will comprise 1,2-napthoquinone or 1,4-naphthoquinones which carry one or more further substitutents.

The naphthoquinone derivative may be a synthetic compound or it may be derived from a natural source. For instance, the active component may comprise an isolated extract from a species of Calceolaria plant for example Calceolaria sessilis, Calceolaria andina or Calceolaria glabrata var. meyenenis which are known to contain naphthoquinone derivatives.

Examples of suitable compounds are described for instance in WO 97/16970, WO 95/32176, U.S. Pat. No. 4,970,328, U.S. Pat. No. 4,929,642, WO96/21355, WO96/21354, WO97/02271 and EP1051909 and these are incorporated herein by way of reference.

Suitable further substituents as defined above include, for instance, hydroxy, alkoxy, aryloxy, aralkyloxy, alkanoyloxy, alkylsulphonyloxy, arylsulphonyloxy, alkyl, alkenyl, halogen, nitro, cyano, amino, mono- or di-alkylamino, alkoxycarbonyl, carboxyl, alkanoyl, alkylthio, alkylsulphinyl, alkylsulphonyl, carbamoyl, alkylamido, cycloalkyl, aryl, aralkyl; wherein any alkyl, alkenyl or aryl groups or moieties within the groups may be optionally substituted by one or more halo, trifluoromethyl, trifluoromethoxy, trifluoromethylsulphenyl, trifluoromethylsulphonyl, trimethylsilyl, or cyclohexyl which is optionally substituted by methyl, trifluoromethyl or trimethylsilyl.

Alternatively, substituents on adjacent positions on a naphthoquinone ring can be joined together to form an optionally substituted ring which may be saturated or unsaturated, and may contain one or more heteroatoms selected from oxygen, sulphur and nitrogen. The ring suitably comprises from 3 to 7 atoms, for instance, 5 atoms, and in particular is a fused tetrahydrofuran ring. Suitable substitutents for a ring formed in this way may include one or more alkyl groups such as methyl. A particular example of such a compound is dunnione, as described in WO 97/16970.

As used herein, the term “alkyl” refers to straight or branched chains containing from 1 to 20, suitably from 1 to 13 carbon atoms. The term “alkenyl” refers to straight or branched chains of from 2 to 20, suitably from 2-13 carbon atoms. The term “aryl” refers to aromatic groups such as phenyl or naphthyl, and “aralkyl” refers to alkyl groups carrying an aryl substituent such as benzyl. The term “halo” includes chloro, bromo or fluoro.

Particular naphthoquinone derivatives are 1,4-napthoquinone derivatives of general formula (I)

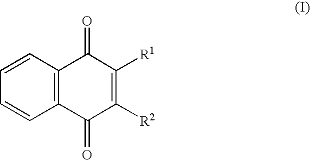

where R¹ is selected from an optionally substituted alkyl group, a hydroxy group or a group —OCOR⁴ where R⁴ is selected from hydrogen, C₁₋₁₂alkyl, C₁₋₁₂haloalkyl, C₁₋₁₂hydroxyalkyl, C₁₋₁₂carboxyalkyl, phenyl or benzyl.

In particular, R¹ is suitably selected from hydroxy of a group —OCOR⁴. Preferred groups R⁴ are hydrogen, C₁₋₆alkyl, C₁₋₆haloalkyl, phenyl or benzyl.

R² is, in particular, is an alkyl, or alkenyl group as defined above, which may be optionally substituted, in particular with a group silicon containing group such as —Si(R⁵R⁶R⁷) where R⁵, R⁶ and R⁷ each represent a C₁₋₄alkyl group, such as methyl.

Particular preferred naphthoquinone derivatives are compounds of formula (III), (IV) or (V) as set out below (and as described in Pest Management Science, 2001, 57 (8) p749-50), or a combination of such compounds.

Compound No.

| | |
|---|---|
| III | 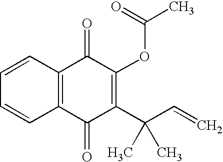 |
| IV | 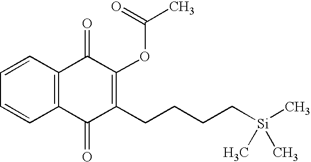 |
| V | 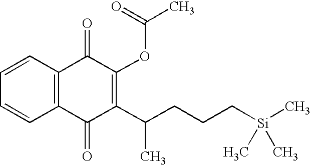 |

Most preferably the naphthoquinone derivative is compound (V) shown above.

Naphthoquinone derivatives such as those described above have been found to be very effective at killing pests, for example Bemisia tabaci (tomato plant pest), Psoroples cuniculi (rabbit ear canker mite), Dermanyssus gallinae (poultry red mite), Psoroples ovis (Sheep scab mite), Musca domestics (housefly) and Blatella germanica (German cockroach).

These chemicals are however photo labile to varying degrees and therefore in there natural state, degrade in UV light. The use of conventional UV protectants either alone or in combination with free radical scavengers(such as bis(2,2,6,6-tetramethyl-4-piperidyl)sebecate and 8-acetyl-3-dodecyl-7,7,9,9-tetramethyl-1,3,8-triazaspiro(4.5)decane-2,5-dione) and/or antioxidants (such as dibutylhydroxy toluene [BHT]) failed to prevent photodegradation of these compounds.

The microcapsules, however, suitably further comprise a dye which can absorb UV light allowing agrochemicals such as those described above to be delivered using microcapsules where previously microcapsule delivery of U.V. labile compounds would not have been effective.

The microcapsules can be formed from any suitable substance, for example gelatine, polyurethane, polyamide, polyurea, polyester or a biodegradable polymer for example Poly-lactide (PLA), but most preferably are comprised of gelatine or polyurethane.

They may be prepared using any conventional method, such as the complex coacervation method or the interfacial polymerisation method. These methods are carried out in the presence of the active component and the particular matter so that the active component becomes encapsulated within the microcapsules and the particulate matter becomes located in the wall of the microcapsule. The encapsulation may also be carried out in the presence of a dye, so that it may also be incorporated into the microcapsule, either encapsulated within them, or in the surface layer.

Alternatively or additionally, dye may be applied subsequently to the prepared microcapsules.

The microcapsules suitably have an average diameter of less than 80 μm, but preferably have an average diameter of less than 60 μm. More preferably the microcapsules have an average diameter of 50 μm. More preferably the microcapsules have an average diameter of 55 μm. Most preferably the microcapsules are between 3 and 35 μm in diameter.

According to a second aspect of the present invention there is provided a pharmaceutical, agrochemical or cosmetic formulation comprising a microcapsule as described above, in combination with a pharmaceutically, veternarily, cosmetically or agriculturally acceptable carrier, diluent or excipient.

The formulation preferably comprises a dye as described above. The dye preferably coats the surface of the microcapsule but may instead or additionally be dispersed throughout the microcapsule. The presence of the dye may, in some circumstances, reduce any phytotoxicity of the formulation in certain plants.

Formulations of this type, for example, a pesticide formulation, a sun tan lotion formulation, a fragrance formulation or a topical medicine formulation, when applied, would leave a mark on the skin of the animal such as human to whom it is applied, giving a visual indication of the areas of skin to which the formulation has and has not been applied.

Suitable carriers, diluents or excipients include solid or liquid excipients and will be selected in accordance with routine practice in the particular field. For instance, agrochemical formulations will generally further comprise an agriculturally acceptable carrier or diluent as is known in the art. Concentrates in the form of solids or liquids may be prepared, which require dilution in water prior to application, for example by spraying.

The formulation can be formed into, for example, water dispersible granules, slow or fast release granules, soluble concentrates, oil miscible liquids, ultra low volume liquids, emulsifiable concentrates, dispersible concentrates, oil in water, and water in oil emulsions, micro-emulsions, suspension concentrates, aerosols, capsule suspensions and seed treatment formulations.

The formulation type chosen in any instance will depend upon the particular purpose envisaged and the physical, chemical and biological properties of the formulation.

Granules may be formed either by granulating microcapsules as described above and one or more powdered solid diluents or carriers. One or more other additives may also be included in granules, for example an emulsifying agent, wetting agent or dispersing agent.

Dispersible Concentrates may be prepared by mixing microcapsules as described above in water or an organic solvent, such as a ketone, alcohol or glycol ether. These dispersions may contain a surface-active agent.

Suspension concentrates may comprise aqueous or non-aqueous suspensions of microcapsules as described above. Suspension concentrates may be prepared by combining microcapsules in a suitable medium, optionally with one or more dispersing agents, to produce a suspension of the microcapsules. One or more wetting agents may be included in the suspension and a suspending agent may be included to reduce the rate at which the microcapsules settle.

Aerosol versions of the formulations may further comprise a suitable propellant, for example n-butane. Suitably microcapsules as described above may also be dispersed in a suitable medium, for example water or a water miscible liquid, such as n-propanol, to provide formulations for use in non-pressurised, hand-actuated spray pumps.

Agrochemical formulations may further include one or more additives to improve the biological performance, for example by improving wetting, retention or distribution on surfaces; resistance to rain on treated surfaces; or uptake or mobility of the microcapsules. Such additives include surface active agents, spray additives based on oils, for example certain mineral oils or natural plant oils (such as soy bean and rape seed oil), and blends of these with other bio-enhancing adjuvants.

Formulations as described above may also be adapted for use as a seed treatment.

Wetting agents, dispersing agents and emulsifying agents may be surfactants of the cationic, anionic, amphoteric or non-ionic type, as is known in the art.

Suitable suspending agents which may be included in the formulations include hydrophilic colloids (such as polysaccharides, polyvinylpyrrolidone or sodium carboxymethylcellulose) and swelling clays (such as bentonite or attapulgite).

The formulations may also contain other compounds having biological activity, for example micronutrients or other agrochemicals having similar or complementary activity.

Pharmaceutical compositions comprising formulations as described above may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular dosing or as a suppository for rectal or vaginal dosing.

The pharmaceutical compositions may be obtained by conventional procedures using conventional pharmaceutical excipients, well known in the art.

Aqueous suspensions suitably will contain the microcapsules together with one or more suspending agents, dispersing or wetting agents. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the microcapsules in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These pharmaceutical formulations may be preserved by the addition of an anti-oxidant such as ascorbic acid.

Topical formulations, such as creams, ointments, gels and aqueous or oily solutions or suspensions, may generally be obtained by mixing a microcapsule as described above with a conventional, topically acceptable, vehicle or diluent using conventional procedure well known in the art.

For further information on Formulation the reader is referred to Chapter 25.2 in Volume 5 of Comprehensive Medicinal Chemistry (Corwin Hansch; Chairman of Editorial Board), Pergamon Press 1990.

The amount of active component that is combined with one or more excipients to produce a single dosage form will necessarily vary depending upon the host treated and the particular route of administration.

Generally agrochemical formulations will be delivered using conventional large scale spray equipment. However, for certain horticultural or pharmaceutical applications, formulations may be incorporated into suitable delivery devices such as atomisers, nebulizors or spray guns.

According to a third aspect of the present invention there is provided a delivery device, such as an atomiser, nebulizor or spray gun containing a microcapsule or formulation as described above. The atomiser, nebulizor or spray gun can be used to apply the microcapsule or formulation to its intended target.

For example if the microcapsules contain a pesticide or insecticide the atomiser, nebulizor or spray gun can be used to apply the microcapsule or formulation to a plant, animal or its environment to provide protection from pests. Formulations may be in the form of a dispersion of a solid in a gas or liquid. These may be prepared for example, from suspensions of the microcapsules in a liquid such as water, using a device such as a nebulizer, or from dry powders. In the case of a nebulized aerosol, the dispersion comprises essentially wet microcapsules in air.

According to a fourth aspect of the invention there is provided a method of protecting a plant, said method comprising administering to the plant or its environment a formulation comprising a microcapsule as described above and wherein the active component is an agrochemical for example a pesticide such as an insecticide.

Preferably the agrochemical is a naphthoquinone derivative. If desired, a dye as described above may be administered separately. Suitably however, the dye, where present, is included in the microcapsule, and the administration takes place in a single step.

The formulation may be applied by any of the known means of applying agrochemical compounds. For example, it may be applied, formulated or unformulated, to the pests or to a locus of the pests (such as a habitat of the pests, or a growing plant liable to infestation by the pests) or to any part of the plant, including the foliage, stems, branches or roots, to the seed before it is planted or to other media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapour or applied through distribution or incorporation of the formulation in soil or an aqueous environment.

Formulations as described above may be sprayed onto vegetation using electrodynamic spraying techniques or other low volume methods, or applied by land or aerial irrigation systems.

Formulations as described above may be supplied in the form of a concentrate, the concentrate being added to water before use. These concentrates, are often required to withstand storage for prolonged periods and, after such storage, to be capable of addition to water to form aqueous preparations which remain homogeneous for a sufficient time to enable them to be applied by conventional spray equipment. Such aqueous preparations may contain varying amounts of the formulation (for example 0.0001 to 10%, by weight) depending upon the purpose for which they are to be used.

According to a fifth embodiment of the invention there is provided a method for producing a microcapsule having a permeable wall comprising forming a microcapsule in the presence of an active component and particulate matter as defined above. Preferably the particulate matter comprises a nano particle and/or an ethylcellulose microsphere, and/or a silica coated particle, such as a silica coated titanium oxide. Suitably the particulate matter such as the ethylcellulose microsphere includes a leachable material.

Where the particulate material includes a leachable material, such as Eudragit® E100, the method suitably includes a further step of leaching said material, either before or after preparation of the microcapsule.

Preferably the active component is an agrochemical, for example a pesticide such as compound (V). Preferably the surface of the microcapsule is dyed and/or a dye is incorporated into the microcapsule during the preparation thereof.

The work revealed here has shown that titanium dioxide and particularly silica coated titanium dioxide particles are at least partially phytotoxic to some plants. This may be due to the desiccating effect caused by the silica on the surface of the titanium dioxide particles and the photocatalytic effect of titanium dioxide. This finding opens up the possibility that these particles could be used as herbicides, in particular as broad-spectrum dessicants.

Thus according to yet a further aspect of the invention, there is provided a method for killing or controlling plants by application of titanium dioxide particles, and particularly silica coated titanium dioxide particles thereto.

These particles will generally be applied in the form of a herbicidal composition, in which they are combined with agriculturally acceptable carriers and such compositions form yet a further aspect of the invention.

The invention will now be particularly described by way of example and with reference to the following Figures.

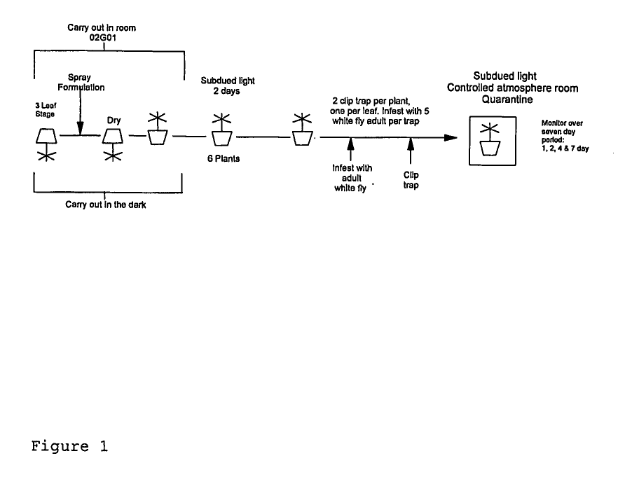

FIG. 1, shows the schematic protocol for the Bioassay.

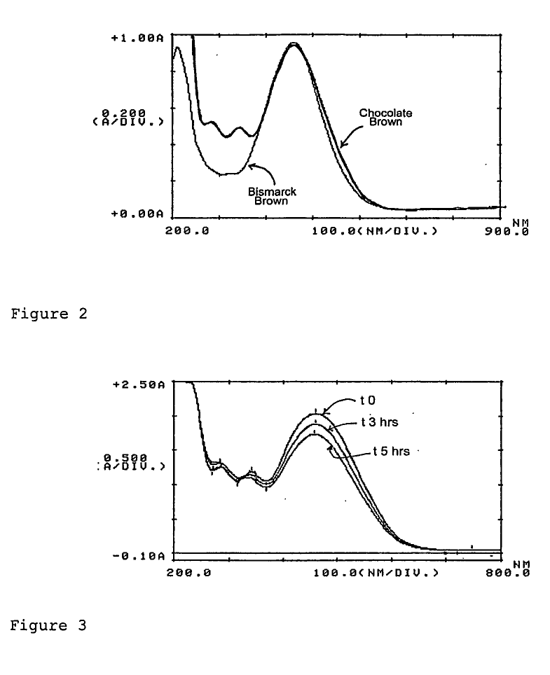

FIG. 2, shows UV absorption spectra of Chocolate Brown and Bismarck Brown R.

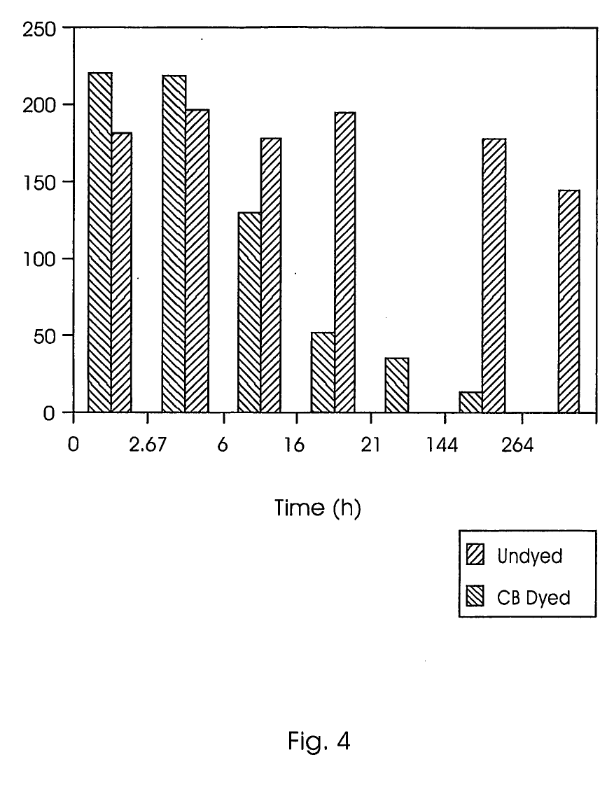

FIG. 3, shows Chocolate Brown irradiated with 254 nm UV light.

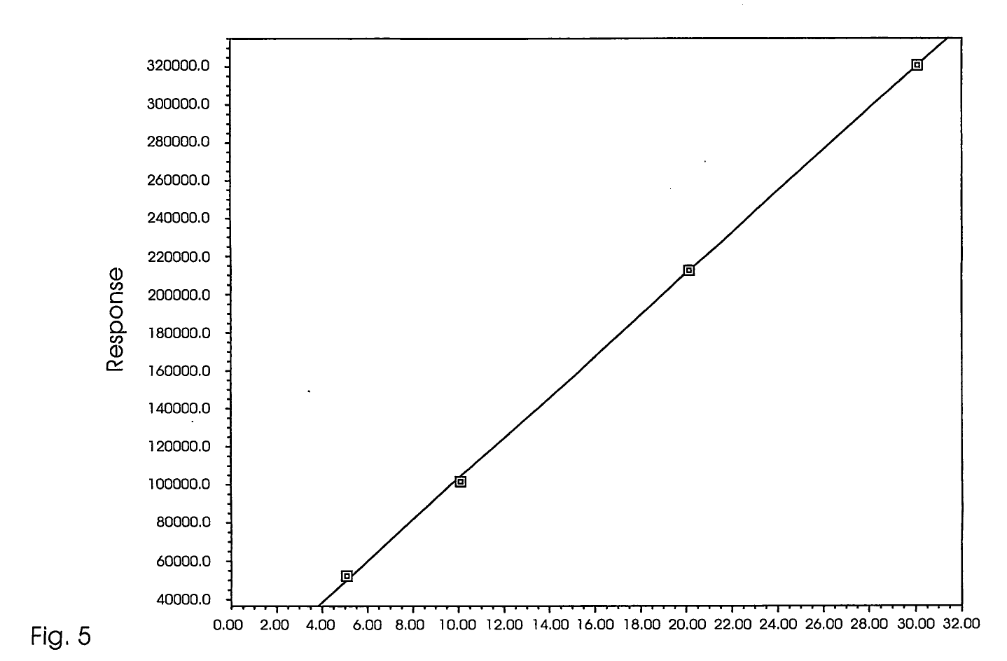

FIG. 4, shows the stability of Compound (V) in undyed and Chocolate Brown (CB) dyed impervious gelatine microcapsules exposed to daylight.

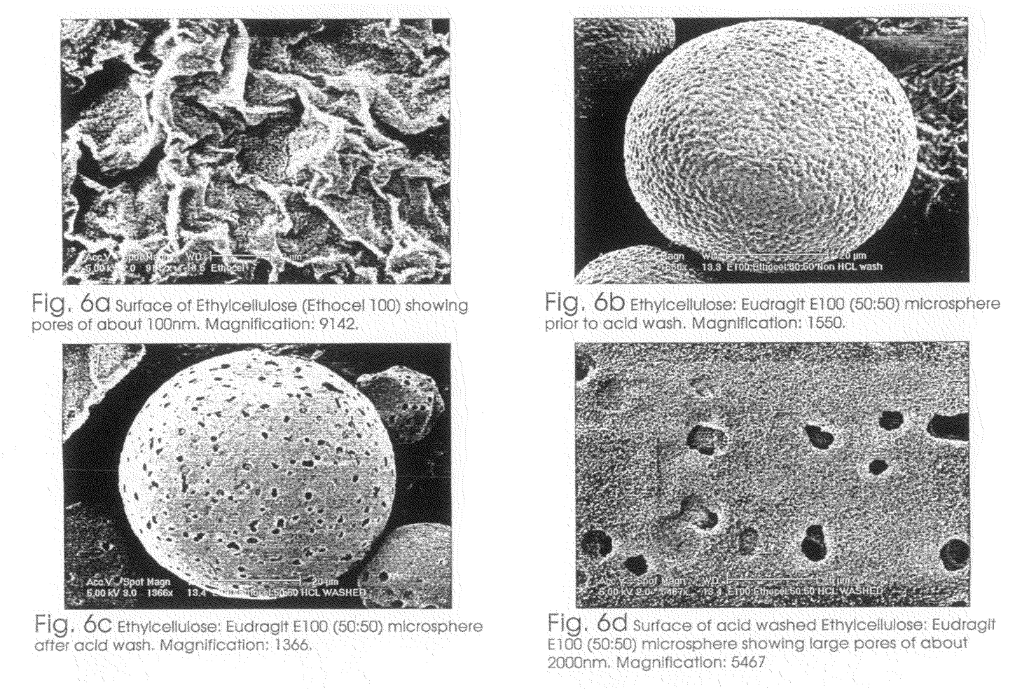

FIG. 5 shows a calibration curve for quantification of Compound (V) by HPLC. Correlation coefficient(R²)=0.9996

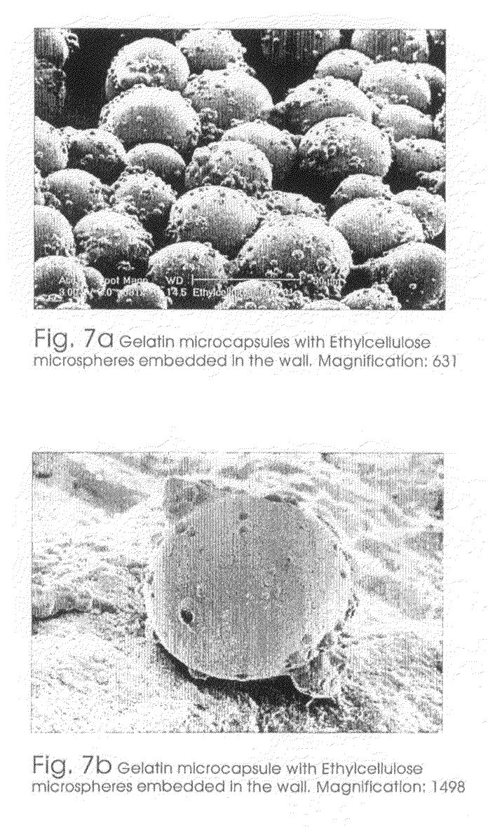

FIG. 6a to 6d show SEM micrographs of various microcapsules showing their surface morphology.

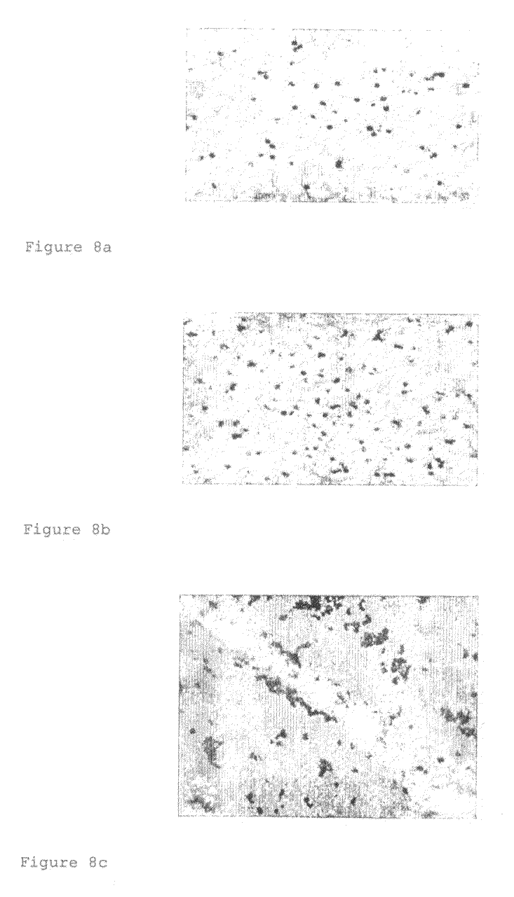

FIG. 7a to 7b show SEM micrographs of Ethylcellulose embedded in the walls of microcapsules.

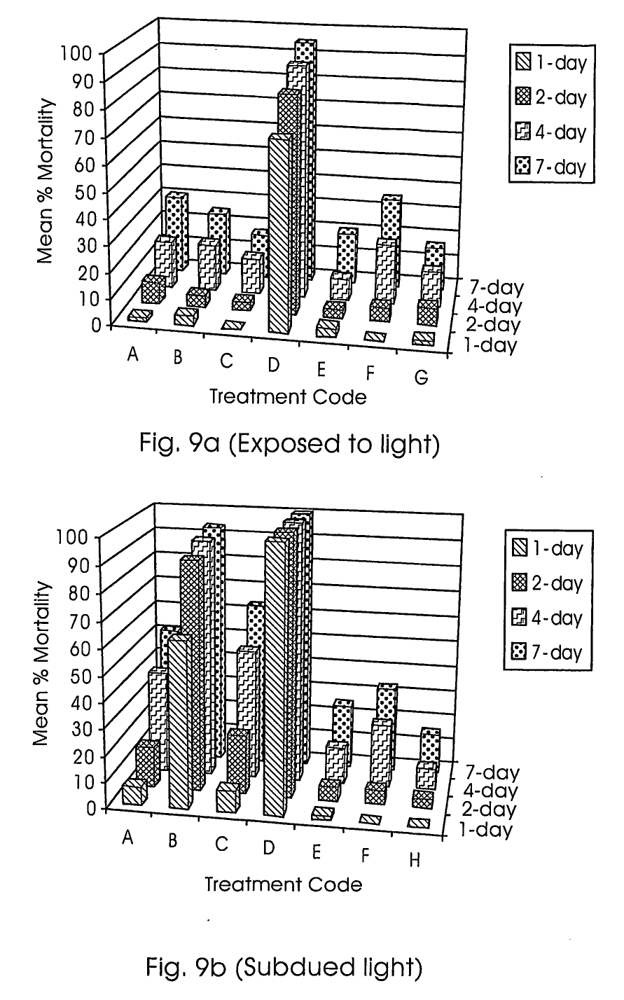

FIGS. 8a and 8b show photomicrographs of capsule distribution pattern obtained with (a) 1/8 and (b) 1/4 dilution of spray solution on filter paper.

FIG. 8c shows a photomicrograph of capsule distribution pattern obtained with 1/6 dilution of spray solution on the abaxial surface of tomato leaf.

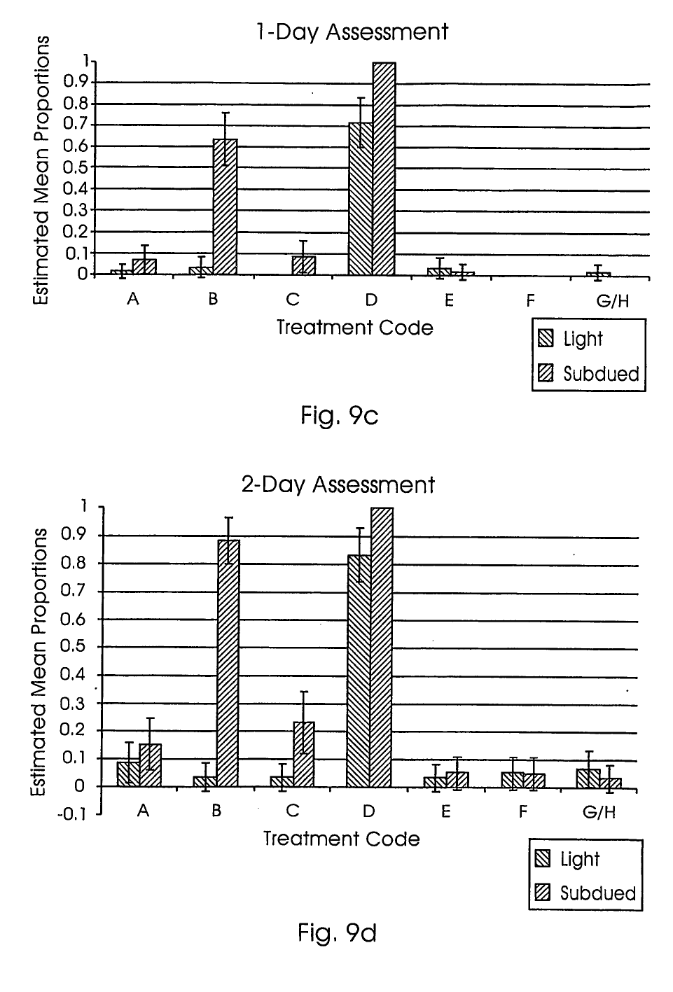

FIG. 9a, shows mean mortality of B. tabaci in the Bioassay, exposed to daylight.

FIG. 9b, shows mean mortality of B. tabaci in the Bioassay, exposed to subdued light.

FIG. 9c, shows mean mortality of B. tabaci after 1 day in the Bioassay.

FIG. 9d, shows mean mortality of B. tabaci after 2 days in the Bioassay.

FIG. 9e, shows mean mortality of B. tabaci after 4 days in the Bioassay.

FIG. 9f, shows mean mortality of B. tabaci after 7 days in the Bioassay.

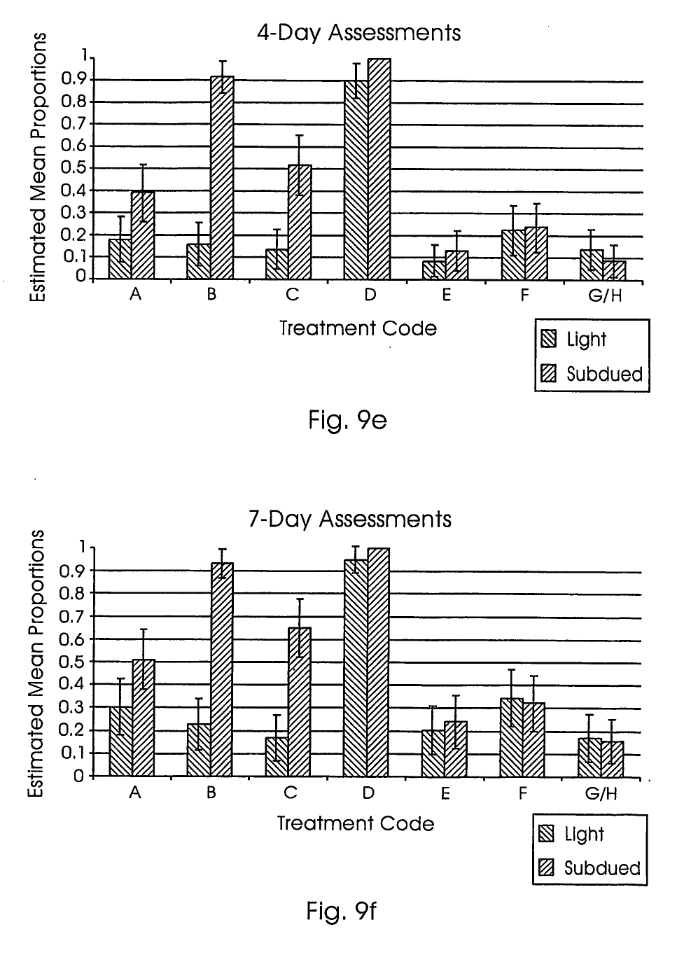

FIG. 10a, shows an SEM micrograph of gelatine microcapsule (mean diameter 50 μm) with Ti-Pure® R-931 incorporated in the wall.

FIG. 10b, shows an SEM micrograph of artificially broken gelatine capsule showing the distribution of Ti-Pure® R-931 in the wall.

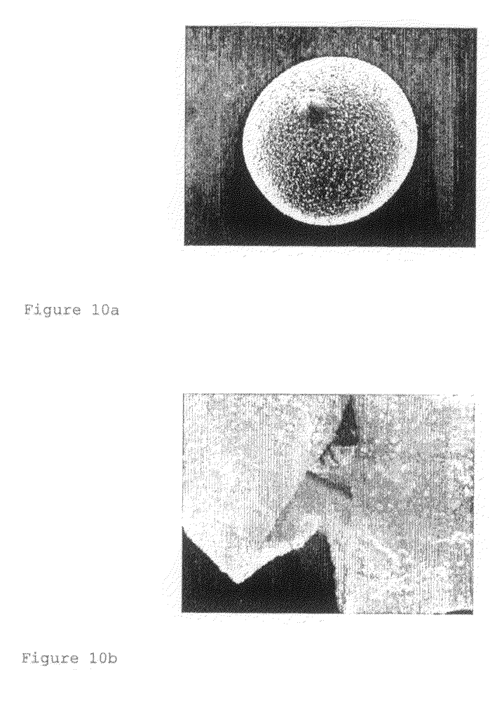

FIG. 11, shows a photograph of tomato plants two days after treatment with various Ti-Pure® R-931 incorporated gelatine microcapsule formulations (A-in middle with label hidden, B, C, D & E) as per the Bioassay. F-no treatment (absolute control).

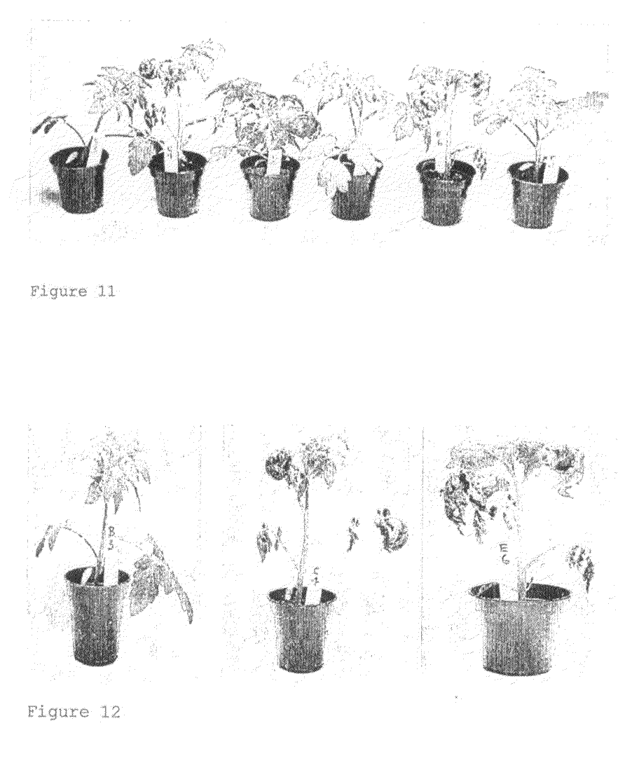

FIG. 12, shows photographs of tomato plants two days after treatment with various R— Ti-Pure® 931 incorporated gelatine microcapsule formulations as per Bioassay 2. Treatment B: Ti-Pure® R-931+COMPOUND (V) (mean diameter of microcapsules: 50 μm) Chocolate Brown dyed.

Treatment C: Ti-Pure® R-931+COMPOUND (V) (mean diameter of microcapsules: 25 μm) undyed Treatment E: Ti-Pure® R-931 (mean diameter of microcapsules: 50 μm) undyed.

The following materials and methods were used during the experiments described below.

Compounds (III)-(V) were supplied by IACR-Rothamsted. Porcine gelatine (Type A, Isoelectric point 8) omniTechnik Microverkapselungs-Gmbh (Germany), Ethocel® 100 (Ethylcellulose, a Dow Chemical Company product) Univar (Croydon), Eudragit® (E100) Rohm (Germany), Exxsol® (D 100) and Solvesso® (100) ExxonMobil (Belgium), Desmondur VL (Diphenylmethane-diisocyanate, MDI) Bayer (Germany), Ti-Pure® R-931 (Titanium dioxide) DuPont (Belgium) and Chocolate Brown HT (Brown 3, CI-20285, E155) WS Simpson (London) were obtained as gifts. All other dyes and chemicals were purchased from Sigma-Aldrich chemical company (Dorset). Laboratory sprayer (Ecospray®, Labo-Chemie-France) was purchased from Rotec Scientific Limited (Milton Keynes). The results of the bioassays were analysed using Genstat 5th edition, release 4.2.

### Ultraviolet Spectroscopy.

Ultraviolet (UV) absorbance spectra were recorded on a dual beam spectrophotometer (Shimadzu, UV-160A) using matched pair of quartz cuvettes of 1 cm path length. Spectra of all water-soluble dyes were obtained as aqueous solutions in double distilled water. Spectra of all non water-soluble compounds were obtained as solution in appropriate solvent. Typically spectra of dyes were recorded over 800-200 nm range. (See FIGS. 2 and 3)

### High Performance Liquid Chromatography (HPLC).

The HPLC system was from Waters comprising of two 510 pumps, a 717 plus Autosampler, a System Interface Module, a Lambda-Max 480 detector and Millennium Chromatography Manager software. Chromatography was achieved on a Zorbax ODS 5 μm C18 analytical column of dimension 4.6×250 mm internal diameter maintained at 35° C. Mobile phases were unmodified water (Milli-Q grade) in reservoir A and unmodified acetonitrile in reservoir B. Linear gradient elution was used with 70 to 90% B over the first 10 minutes, then 100% B for 3 minutes and returning to 70% B over 4 minutes. Injection cycle time was 20 minutes with a flow rate of 2 ml/min. The samples were either dissolved in acetonitrile or diethyl ether and 10 μl volume injected on to the column which was maintained at 35° C. Prior to use, mobile phase were degassed under vacuum with sonication and continuously sparged with helium. The naphthoquinones were detected by measurement of UV absorbance at 269 nm.

### Scanning Electron Microscopy.

Microcapsule specimens were mounted on aluminium stubs and coated with gold in an Emscope SC500A sputter coater. Specimens were examined and photographed with a Phillips XL20 scanning electron microscope.

### Identification of Dyes Suitable for Photostabilisation of Compound (V).

Dyes with absorption spectra similar to Bismarck Brown R were selected as potential candidates for dying microcapsules since Bismarck Brown was found to absorb UV light. Aqueous solutions of the dyes were prepared, an aliquot of each solution was transferred to a quartz cuvette and the UV absorbance spectrum recorded. The cuvette containing the solution was then irradiated with 254 nm UV light, with the clear surface of the cuvette facing the radiation source, for various time periods and the spectra recorded again.

### Preparation of Microspheres.

Microspheres containing a mixture of ethylcellulose and Eudragit® E 100 (3:1) were made by emulsifying a solution of the polymer mixture into an aqueous solution of gelatine.

Typically, 1 g of polymer mixture was dissolved in 40 ml of dichloromethane at room temperature. The polymer solution was dispersed in 130 ml of 2% (w/v) aqueous gelatine solution at 30° C. with an Ultra Turrax® homogeniser to give about 20 μm droplets and the agitation continued through out the rest of the procedure. The dispersion was warmed in a water bath to 40° C. and maintained at that temperature for four hours. The system was then allowed to cool to room temperature, the resultant microspheres washed thoroughly with water and resuspended in 3 ml of water. The polymeric microspheres had a mean size of about 5 μm diameter.

Eudragit® E 100 polymer was leached from the ethylcellulose/Eudragit® E 100 microspheres, by suspending them in 1M hydrochloric acid to provide porous ethylcellulose microspheres.

### Preparation of Gelatine Microcapsules.

Gelatine microcapsules were produced by the complex coacervation method. Typically, the pH of 140 ml of 1.33% (w/v) aqueous gelatine (type A with isoelectric point 8) solution, maintained at 45° C., was adjusted to 6.25 with 10% (w/v) sodium hydroxide. 15 ml of dibutylsebecate containing 1% by volume Span 859, pre warmed to 45° C., was added to the gelatine solution and dispersed with a mechanical stirrer. The droplet size of the dibutylsebecate dispersion was adjusted and the agitation continued through out the rest of the procedure. 3 ml of a 70% by weight aqueous dispersion of Ti-Pure® R-931 was added to the dibutysebecate dispersion, followed by drop wise addition, over 10 minute period, of 30 ml of 0.5% by weight aqueous carrageenan (Type 1) solution at 45° C. The system was then allowed to cool to room temperature slowly. Once the system had reached room temperature, it was chilled to 4° C. using an ice bath and maintained at that temperature for one hour. 5 ml of 25% by weight aqueous gluteraldehyde solution was added to the chilled system and maintained for a further one hour at 4° C. The ice bath was then removed, the system allowed to warm up and maintained at room temperature for about 18 hours. (See FIGS. 6a to 6d)

Compound (V) when present, was encapsulated as a solution in 15 ml of either Exxsol® D 100 or dibutylsebecate. Typically the microcapsules had a mean size of either 25 μm or 50 μm diameter.

Microencapsulation was also carried out, in the presence the microspheres or nano particles of Ti-Pure® R-931, to incorporate the particulate matter into the wall of the capsules to make them permeable. The capsules were harvested either as a slurry or wet cake. The microcapsules contained Span 85 (sorbitan trioleate) as a surfactant, to promote the translocation of COMPOUND (V) into whitefly. Appropriate placebo microcapsules were produced to carry out preliminary tests and to act as controls in bioassay. (See FIGS. 7a and 7b)

The presence of Ti-Pure® R-931 has an additional advantage of inhibiting aggregation of the dispersed droplets during production of the gelatine microcapsules. Sometimes during the preparation of the microcapsules, especially capsules below 50 microns, the dispersed droplets are encapsulated as aggregates resulting in bigger capsules. Ti-Pure® R-931 inhibits this aggregation enabling discrete microcapsules of below 10 microns to be formed. These smaller microcapsules are easier to apply to a plant or an animal by spraying, as they do not clog up the nozzle of any spraying device.

### Preparation of Polyurethane Microcapsules.

Polyurethane microcapsules of COMPOUND (V), as a solution in Solvesso® 100, were produced by the interfacial polymerisation method using Desmondur VL and ethyleneglycol in the organic and aqueous phase respectively. Typically, 15 ml of a 6.7% by volume solution of Desmondur VL in Solvesso® 200 was dispersed in 120 ml of 5% (w/v) solution of gum acacia at room temperature. The droplet size of the dispersion was adjusted and the agitation continued through out the rest of the procedure. 5 ml of ethyleneglycol was added dropwise to the dispersion and the system was warmed in a water bath to 60° C. and maintained at that temperature for 18 hours. The system was then allowed to cool to room temperature and the resultant microcapsule slurry was diluted with water as requited.

Typically the microcapsules had a size range of 5 to 30 μm in diameter. Encapsulation was also carried out in the presence of Chocolate Brown dissolved in the aqueous phase. Appropriate placebo microcapsules were produced to act as controls.

### Photo Stabilisation Study Using Chocolate Brown Dye.

A batch of gelatine microcapsules containing 300 mg of COMPOUND (V) in 15 ml of Exxsol® D100 was produced.

The capsule slurry was washed repeatedly with water to remove debris and filtered to obtain a wet cake. An aliquot of the wet cake (11.3 g) was made up to 50 ml and dyed brown with Chocolate Brown (500 mg, equivalent to 10 mg/ml solution). Aliquots (200 μl) of microcapsule slurry of brown and undyed capsules were applied to glass microscope slides in duplicate. The slurry from each batch was spread to form a monolayer of microcapsules on each slide. The slides were air dried in a dark at room temperature (≅21° C.) and exposed to daylight on a south-facing windowsill for various time periods. Two slides from each batch were analysed per time point post of exposure. Unexposed slides stored in the dark were used as time zero reference.

The contents of the capsules were extracted by rupturing the capsules, by rolling a glass rod on the slides, and washing both the rod and the slide with diethyl ether. The extracts were made up to 10 ml and assayed by HPLC. Examination of the slides under the microscope showed that the capsules were all broken and had released their contents.

### Bioassay.

Tomato plants used in the bioassay were grown in controlled glasshouse cubicles at 20° C., 12 h Light: 12 h Dark (12L: 12D) light regime, using 400 watt holophane daylight bulbs, to the third true leaf stage (approximately five weeks old from sowing). Whiteflies were cultured on poinsettia (Euphorbia pulcherrima) maintained at 22° C., 16L: BD light regime and 65% relative humidity. Adults were removed from stock culture when required for infestation of test plants. Bioassays were carried out “blind”, i.e. all treatments were unknown to the investigators throughout the trial. Phytotoxic effects, such as scorching, leaf distortion necrosis or necrotic lesions were assessed at one week and one month intervals. Any signs were noted at each assessment period and photographs were taken of each treatment set. Any plant showing signs of phytotoxicity was photographed.

### Determination of the Optimum Capsule Density in the Spray Solution and Preliminary Phytotoxicity Studies.

Aliquots of Chocolate Brown dyed placebo gelatine microcapsule (50 μm mean diameter) wet cake were made up to 50 ml with water to obtain 1/8, 1/4 and 1/2 dilution of capsules in a laboratory sprayer (Ecospray®). Filter paper and both surfaces of tomato leaves were sprayed with the diluted capsule slurry.

The sprayed objects were allowed to air dry and the distribution of the capsules monitored both by naked eye and under a microscope. Representative areas (2 cm²) were cut from the filter paper sprayed with 1/8 and 1/4 dilution of capsule slurry, sandwiched between two glass slides and viewed under the microscope. The number of capsules present in the field of view (2.27 mm²), at randomly selected areas of the filter paper, were counted. The sprayed tomato leaves were examined qualitatively under the microscope.

Either the top or the abaxial leaf surface of tomato plants was sprayed with either 1/8 or 1/4 dilution of capsule slurry in duplicate. Two plants were sprayed on both surfaces with 1/4 dilution of capsule slurry. All plants were transferred to the glasshouse and monitored for phytotoxic effects.

The Bioassay was carried out according to the schematic protocol shown in FIG. 1. Only gelatine microcapsule formulations with Ti-Pure® R-931 incorporated in the capsule wall were used in the bioassay. In this case the formulations used can be summarised as follows:

| | Material | | |
|---|---|---|---|
| | incorporated into | | |
| Formulation | gelatine | Active | Dyed/undyed |
| A | Ti-Pure ® R931 | Compound V | undyed |
| | | (50 μm) | |
| B | Ti-Pure ® R931 | Compound V | dyed |
| | | (50 μm) | |
| C | Ti-Pure ® R931 | Compound V | undyed |
| | | (25 μm) | |
| D | Ti-Pure ® R931 | Compound V | dyed |
| | | (25 μm) | |
| E | Ti-Pure ® R931 | control | undyed |
| | | (25 μm) | |
| F | No treatment | | N/A |
| | absolute control | | |
| G | Ti-Pure ® R931 | control | dyed |
| | | (25 μm) | |

COMPOUND (V) microcapsule formulations were produced with 300 mg of the compound dissolved in 15 ml of dibutylsebecate containing 1% (v/v) Spans 85. The microcapsule slurries were diluted to give 1000 ppm of COMPOUND (V) in 1/6^{th} dilution of capsules, in the final spray solutions. The final spray

10. solutions also contained 0.33% (v/v) Tween® 20 [POE (20) sorbitan monolaurate] as surfactant in the aqueous medium. The microcapsules in formulations B, D and G were dyed with 6.6 mg/ml solution of Chocolate Brown.

Formulations A and B had a mean-capsule size of 50 μm diameter and all of the others were 25 μm.

The abaxial surface of the leaves of 6 tomato plants per formulation were sprayed with the various formulations, allowed to dry in the dark and transferred to a controlled environment room. Six untreated plants were used as absolute control (F). It was noticed that the plants sprayed with undyed microcapsules showed phytotoxic effects and these were eliminated from the bioassay. All remaining plants in the controlled environment room were infested with whiteflies as per the protocol in FIG. 1.

Mortality rate of whiteflies in the clip cages were monitored over a seven-day period at 1, 2, 4 and 7 days post infestation.

### Results and Discussion

Dyes which have similar UV absorption spectra to that of Bismarck Brown are given in Table 1 below.

TABLE 1
UV protection dyes for 1,4-naphthoquinone pesticides

| | | WATER | |
|---|---|---|---|
| | | SOLUBILITY | |
| NAME | λmax | (mg/ml) | REMARKS |
| Acid Orange 51 | 446 (water) | 30 | Sulphonic acid |
| | | | derivative, |
| Acid Orange 63 | 424 (water) | 50 | Sulphonic acid |
| | | | derivative |
| Acid Orange 74 | 455 (water) | 20 | Sulphonic acid |
| | | | derivative |
| Bismark Brown R | 468 (50% | 70 | Diazo |
| | Ethanol) | | |
| Bismark Brown Y | 457 (50% | 50 | Diazo |
| | Ethanol + HCl) | | |
| Bromocresol | 423 | 6 | Sulphone- |
| Green | (Methanol) | | phthalein |
| Chlorophenol Red | 575 (H2O) | 60 | Sulphone- |
| | | | phthalein |
| | | | pHindicator |
| Chrysoidin | 449 (H2O) | 20 | Monoazo pH |
| | | | indicator |
| Congo Red | 497 | 40 | Diazo pH |
| | (H2O + NaOH) | | indicator |
| m-Cresol Purple | 436 (H2O) | 2 | pH indicator |
| Crocein Orange G | 482 (H2O) | 40 | Monoazo |
| Darrow Red | 502 (50% | 1 | Oxazine |
| | Ethanol) | | |
| Direct Black 22 | 481 (H2O) | ? | Polyazo |
| Ethyl Orange | 474 (H2O) | 100 | Monoazo pH |
| Ethyl Red | 447 (0.1N | 3 | Monoazo pH |
| | NaOH) | | |
| Methyl Red | 493 | 2 | Monoazo pH |
| | (Methanol + | | |
| | HCl) | | |
| Mordant Brown 1 | 373/487 | 60 | Diazo |
| | (H2O) | | |
| Mordant Brown 4 | 500/374 | 60 | Monoazo (hot |
| | (Ethanol) | | water) |
| Mordant Brown 33 | 442 (H2O) | 20 | Monoazo |
| Mordant Brown 48 | 492 (H2O) | 40 | Monoazo |
| Chocolate Brown | 459 (H2O) | 40 | Diazo. |
| | | | Sulphonic acid |
| | | | derivative |
| | | | (Food dye) |

The chemical structure of Bismarck Brown is:—

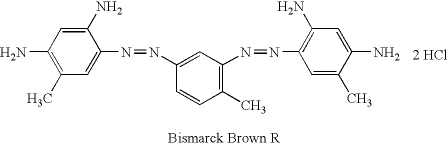

Bromcresol Green, Ethyl Orange, Ethyl Red, Mordant Brown 33, Mordant Brown 48, and Chocolate Brown were selected as candidates for dying microcapsules since these are environmentally acceptable dyes and are therefore preferable to Bismark Brown, which is not environmentally acceptable.

Chocolate Brown was found to have the best spectral characteristics and UV stability when exposed to 254 nm UV irradiation as shown in FIGS. 2 and 3.

Unlike Bismarck Brown, the reductive cleavage of azo bonds in Chocolate Brown does not result in the production of carcinogenic aromatic amines. This is the reason Chocolate Brown can be used as a food colorant. Therefore, Chocolate Brown was selected for dying COMPOUND (V) microcapsules as a preferred dye.

The results of in vitro COMPOUND (V) photostabilisation studies carried out with undyed and Chocolate Brown dyed impervious gelatine microcapsules are shown in FIG. 4. The COMPOUND (V) calibration curve, used in this study, for quantitation of the compound by HPLC, is shown in FIG. 5. Four standard solutions of COMPOUND (V) in acetonitrile, with three replicates per standard, were used to generate the calibration curve.

COMPOUND (V) in undyed capsules degraded progressively on continued exposure to daylight. The amount of COMPOUND (V) in these capsules was reduced to 60% of the initial amount after 6 hours exposure, 23% after 16 hours and only 6% after 40 hours (equivalent to 8 hours daylight exposure over 5 days). In contrast, almost 80% of the initial amount of COMPOUND (V) was present in Chocolate Brown dyed capsules even after 88 hours (equivalent to 8 hours daylight exposure over 11 days) exposure to daylight.

Since gelatine microcapsules are impervious to their contents, they were made more pervious by incorporating particulate matter in the wall. To this end, ethylcellulose, and Eudragit® E100 leached ethylcellulose microspheres were made.

The SEM micrographs of the various microspheres are shown in FIGS. 6a to 6d. The ethylcellulose microspheres have very small pores about 100 nm diameter(FIG. 6A). The acid washed ethylcellulose/Eudragit® E100 microspheres (Ethylcellulose:Eudragit® E100 [50:50]) have, in addition to the 100 nm diameter pores, a lot of large pores of about 2000 nm diameter(FIG. 6C).

The microspheres were incorporated into the gelatine microcapsule wall by carrying out the encapsulation in the presence of a specific type of microsphere dispersed in the aqueous phase.

The SEM micrographs of the gelatine microcapsules with the various types of microspheres embedded in the wall are shown in FIGS. 7a to 7b.

Unlike gelatine, polyurethane did not take up Chocolate Brown dye as effectively. Therefore, an alternative technique of incorporating the dye into the polyurethane wall was carried out. Microencapsulation was carried out with Chocolate Brown dissolved in the aqueous medium, so that the dye could be incorporated into the wall by the chemical reaction between the isocyanate moieties in Desmondur VL and the hydroxy moieties in Chocolate Brown, at the oil/water interface:

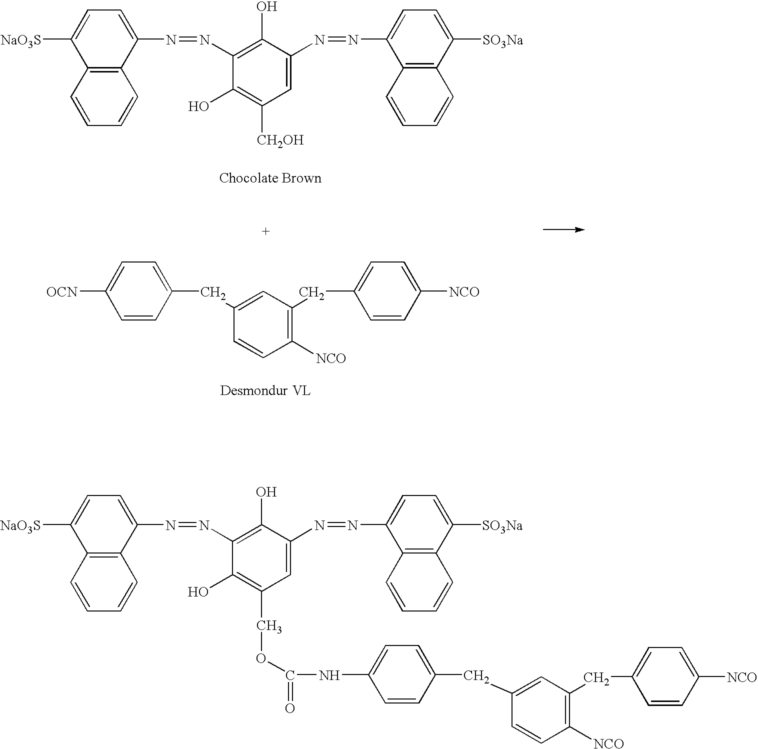

The microcapsules produced, were mostly aggregated and had faintly dyed walls surrounded by a brownish diffuse material. Such particles could be used in formulations according to the present invention, since some dye was incorporated into the walls of the microcapsules.

However, plain polyurethane microcapsules containing COMPOUND (V) in Solvesso® 100 (Desmondur VL does not disolve in Exxsol® D100) were made, suspended in Chocolate Brown solution.

Prior to carrying out the bioassays it was necessary to determine the appropriate capsule density in the spray solution that would optimise the distribution of the capsules on the leaf surface. A microcapsule spray solution with 1/2 dilution of capsules was difficult to spray using the laboratory sprayer, Ecospray®. The capsule distribution pattern obtained with 1/8 and 1/4 dilution of spray solution on filter paper is shown in FIGS. 8a and 8b.

Mean microcapsule distribution of about 1760 and 4490 capsules per cm² were obtained with a 1/8 and 1/4 dilution of capsules respectively in the spray solution. Although, a superior distribution of microcapsules was obtained with a 1/4 dilution, the high density of capsules in the spray solution tended to block the nozzle. Therefore, an intermediate dilution of 1/6 was chosen for carrying out the bioassay.

The distribution of the microcapsules on both surfaces of tomato leaves was not as uniform as that obtained with filter paper. The capsules showed a tendency to accumulate around the vein area, predominantly in small aggregates as shown in FIG. 8c.

Based on these studies, typically, COMPOUND (V) microcapsule slurry containing 300 mg of the compound was diluted to 300 ml to obtain 1/6 dilution of capsules having 1000 ppm of active ingredient in the final spray solution.

In preliminary toxicity evaluation, all tomato plants sprayed with Chocolate Brown dyed placebo gelatine microcapsule, either at 1/4 or 1/8 dilution of capsules, on both surfaces of leaf, showed no phytotoxic effects.

The results of the efficacy evaluation of reformulated COMPOUND (V) against B. tabaci in the bioassay are given in FIGS. 9a to 9f. Mortality of flies in the absolute control (F) remained below 10% over the seven-day monitoring period. Although the mortality (34%) with the placebo formulation (G) was significantly higher than the absolute control, it was not much lower than the mortality (>90%) with formulations B & D. The only difference between B and D was the microcapsule size, 50 μm and 25 μm. The smaller capsule size (25 μm) was used to increase both the volume to surface area and capsule density on the leaf surface. Results showed that no significant improvement was achieved by reducing the capsule size.

These formulations contained fine particles of titanium dioxide, Ti-Pure® R-931, incorporated into the wall of the gelatine microcapsules to make them permeable. Two other types of titanium dioxide, Ti-Pure® R-902 and Ti-Pure® R-960, were also evaluated and found to be incompatible with gelatine solution. Ti-Pure® R-931 has 10.2% amorphous silica coating on the surface, which has an oil absorption capacity of 35.9.

It appears that this coating of silica acts as a wick in transferring the contents of the capsule to the flies on contact. These formulations contained Span® 85 in the organic phase within the capsules and Tween® 20 in the aqueous spraying medium to aid translocation of the active substance to the target and to aid in the wetting and spreading of the formulation on the leaf surface respectively. Dibutylsebecate (DBS, boiling point: 178-179° C./3 mm Hg) was used as the solvent for COMPOUND (V). SEM micrographs of Ti-Pure® R-931 containing gelatine microcapsule are shown in FIGS. 10a and 10b. It is evident from the micrograph of fractured capsule that the particles traverse the wall.

Formulations containing undyed Ti-Pure® R-931 capsules (A, C & E) were found to be highly phytotoxic to tomato plants and were eliminated from the bioassay.

Photographs of phytotoxic effects on tomato plants are shown in FIGS. 11 and 12. Dying the capsules with Chocolate Brown, however, minimised the toxic effect. The capsules employed in the study had the maximum possible loading of Ti-Pure® particles achievable under the microencapsulation conditions used. This was done to maximise the permeability of the capsules to demonstrate the desired effect on the target. Since it has been demonstrated here that Ti-Pure® makes the capsules permeable, it is anticipated that the phytotoxic effects could be eliminated by reducing the loading of Ti-Pure® in the capsules with concomitant dying with Chocolate Brown.

The use of titanium dioxide in microspheres to provide UV protection for bio pesticides (nuclear polyhedrosis virus, which is a stomach poison) has been reported by Bull, D. L.

(Formulations of microbial insecticides: microencapsulation and adjuvants. Formulation and application of microbial insectcides. A symposium at the Annual Meeting of the Entamological Society of America-Honolulu, Hi.; Dec. 1, 1976, Ed. Ignoffo, C. M.; Falcon, L. A., Miscellaneous Publications of the Entamological Society of America, Vol. 10, p 11-20 (1978)). These water-insoluble, but digestible, microsphere formulations were made by a spray-drying, phase-separation process. These workers, however, did not report the type of titanium dioxide used or any phytotoxic effects. Two possible mechanisms may be responsible for the observed phytotoxicity. Ti-Pure® R-931 is coated with a high amount of amorphous silica, which may act by desiccating the leaf tissue. Plants exhibiting phytotoxicity appear to be more susceptible to water stress than the others. Secondly, titanium dioxide is a photo catalyst, which chemically decomposes water molecules into highly reactive hydroxyl ions (OH⁻) under the influence of UV irradiation.

### CONCLUSIONS

Success in producing permeable microcapsules was achieved by incorporating ethylcellulose microspheres into the gelatine microcapsule wall. Similar results were also obtained with polyurethane microcapsule formulations. The incorporation of Ti-Pure® R-931 (titanium dioxide) produced capsules with further improved performance, resulting in more than 95% mortality of whiteflies. Ti-Pure® R-931 incorporated microcapsules were found to be highly phytotoxic to tomato plants. However, dying these capsules with Chocolate Brown. reduces the phytotoxic effects of Ti-Pure® R-931 considerably.

1. A microcapsule having a permeable wall, said microcapsule comprising an active component encapsulated therein, and a particulate matter located in a wall thereof to render the wall permeable.

2. A microcapsule according to claim 1 wherein the particulate matter is a microparticle or a nano particle.

3. A microcapsule according to claim 2 wherein the particulate matter is an inorganic particle such as a metal or an insoluble salt thereof.

4. A microcapsule according to claim 1 wherein the particulate matter is an insoluble polymeric material.

5. A microcapsule according to claim 5 wherein the insoluble polymeric material is alkyl cellulose.

6. A microcapsule according to claim 6 wherein the insoluble polymeric material is ethyl cellulose.

7. A microcapsule according to claim 1 wherein at least some of the particulate matter is coated with silica.

8. A microcapsule according to claim 7 wherein the inorganic particle is silica coated titanium dioxide.

9. A microcapsule according to claim 8 wherein the particulate matter comprises Ti-Pure® R-931.

10. A microcapsule according to claim 1 wherein the particulate matter is combined with a leachable material.

11. A microcapsule according to claim 10 wherein the leachable material is Eudragit® 100.

12. A microcapsule according to claim 1 wherein the microcapsule further comprises a dye.

13. A microcapsule according to claim 12 wherein the dye is incorporated within or located on the surface of the microcapsule.

14. A microcapsule according to claim 12 wherein the dye is Acid Orange 51, Acid Orange 63, Acid Orange 74, Bismark Brown R, Bismark Brown Y, Bromocresol Green, Chlorophenol Red, Chrysoidin, Congo Red, m-crestol Purple, Crocein Orange G, Darrow Red, Direct Black 22, Ethyl Orange, Ethyl Red, Mordant Brown 1, Mordant Brown 4, Mordant Brown 33, Mordant Brown 48 or Chocolate Brown.

15. A microcapsule according to claim 14 wherein the dye is Chocolate brown.

16. A microcapsule according to claim 1 comprising gelatine.

17. A microcapsule according to claim 1 comprising polyurethane.

18. A microcapsule according to claim 1 wherein the active component is a pharmaceutically, cosmetically or veterinarily useful component.

19. A microcapsule according to claim 1 wherein the active component is an agrochemical.

20. A microcapsule according to claim 19 wherein the agrochemical is a pesticide.

21. A microcapsule according to claim 20 wherein the pesticide is a nathoquinone derivative of formula (I) 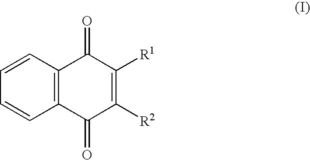
where R¹ is selected from an optionally substituted alkyl group, a hydroxy group or a group —OCOR⁴ where R⁴ is selected from hydrogen, C₁₋₂alkyl, C₁₋₁₂haloalkyl, C₁₋₁₂hydroxyalkyl, C₁₋₁₂carboxyalkyl, phenyl or benzyl.

22. A microcapsule according to claim 21 wherein the compound of formula (I) is a compound wherein R¹ is selected from hydroxy of a group —OCOR⁴, where R⁴ is hydrogen, C₁₋₆alkyl, C₁₋₆haloalkyl, phenyl or benzyl.

23. A microcapsule according to claim 21 wherein R² is an alkyl, or alkenyl group which may be optionally substituted with a group —Si(R⁵R⁶R⁷) where R⁵, R⁶ and R⁷ each represent a C₁₋₄alkyl group, such as methyl.

24. A microcapsule according to claim 1 having an average diameter of less than 60 μm.

25. A microcapsule according to claim 24 having an average diameter of 50 μm.

26. A microcapsule according to claim 24 being between 3 and 35 μm in diameter.

27. A pharmaceutical, veterinary, cosmetic or agrochemical formulation comprising a microcapsule as described in claim 1, in combination with a pharmaceutically, veternarily, cosmetically or agriculturally acceptable carrier, diluent or excipient.

28. A delivery device containing a microcapsule as claimed in claim 1 or a formulation as claimed in claim 27.

29. A method for protecting a plant, said method comprising administering to the plant or its environment a formulation comprising a microcapsule according to claim 19.

30. A method for producing a microcapsule having a permeable wall comprising forming a microcapsule in the presence of an active component and particulate matter.

31. A method as claimed in claim 30 wherein the active component is an agrochemical.

32. A method as claimed in claim 31 wherein the agrochemical is compound (V).

33. A method according to claim 30 wherein the particulate matter is a silica coated metal oxide.

34. A method according to claim 33 wherein the silica coated metal oxide is Ti-Pure® R-931.

35. A method according to claim 30 wherein the particulate matter incorporates a leachable material, and in a preliminary step, the leachable material is removed therefrom.

36. A method according to claim 30 wherein the surface of the microcapsule is dyed and/or a dye is incorporated into the microcapsule during the preparation thereof.

37. (canceled)

38. A method for killing or controlling plants by application of titanium dioxide particles, and particularly silica coated titanium dioxide particles thereto.

39. A herbicidal composition comprising titanium dioxide particles, and particularly silica coated titanium dioxide particles, in combination with an agriculturally acceptable carrier.

40. (canceled)

41. (canceled)
